(19) United States
(12) Patent Application Publication
(10) Pub. No.: US 2026/0272942 A1
(43) Pub. Date: Sep. 17, 2026

(54) COMBINING NIMORAZOLE, CHEMOTHERAPY AND RADIOTHERAPY

(71) Applicant: AFYX THERAPEUTICS A/S, København Ø (DK)

(72) Inventors: Jens OVERGAARD, Aarhus N (DK); Vincent GRÉGOIRE, Lyon (FR); Claus J. MØLLER-SAN PEDRO, Gilleleje (DK)

(21) Appl. No.: 19/575,318

(22) Filed: Mar. 23, 2026

Related U.S. Application Data
(63) Continuation of application No. PCT/DK2025/050034, filed on Mar. 14, 2025.

(51) Int. Cl. A61K 31/5377; A61K 31/282; A61K 31/337; A61K 31/513; A61K 31/7068; A61K 33/243; A61N 5/10; A61P 35/00

(52) U.S. Cl. CPC A61K 31/5377; A61K 31/282; A61K 31/337; A61K 31/513; A61K 31/7068; A61K 33/243; A61N 5/1045; A61P 35/00

(57) ABSTRACT

The present invention relates to the combined use of nimorazole or a pharmaceutically acceptable salt thereof and chemo-radiotherapy. In particular, it relates to the combined use of nimorazole or a pharmaceutically acceptable salt thereof, chemotherapy and radiotherapy for patients suffering from laryngeal, hypopharyngeal and/or oropharyngeal squamous cell carcinoma (SCC).

### CROSS REFERENCE TO RELATED APPLICATIONS

This is a continuation application of PCT Application No. PCT/DK2025/050034, filed on 14 Mar. 2025 and entitled “Combining Nimorazole, Chemotherapy and Radiotherapy”, which is hereby incorporated by reference in its entirety.

### BACKGROUND

Hypoxic tumors have been known for years. The treatment of hypoxic cancers has shown to be difficult presumably because of the hypoxic nature of the tumors which appears to make hypoxic tumors resistant towards cancer treatment methods such as radiotherapy and chemotherapy.

Chemical radio sensitizing agents given concurrently with radiation has been suggested and the compound nimorazole has been tested for concurrent use with radiation for the treatment of head and neck squamous cell carcinomas (HNSCC) cancers in clinical trials.

The International patent application with publication number WO 2014/075692 A1 concerns a tablet comprising nimorazole or a pharmaceutically acceptable salt thereof. The nimorazole tablet may be used for radio sensitizing hypoxic tumor cells.

### SUMMARY

The present invention relates to the combined use of nimorazole and chemo-radiotherapy. In particular, it relates to the combined use of nimorazole or a pharmaceutically acceptable salt thereof, chemotherapy and/or radiotherapy for patients suffering from laryngeal, hypopharyngeal and/or oropharyngeal squamous cell carcinoma (SCC).

It has surprisingly been discovered that the administration of nimorazole or a pharmaceutically acceptable salt thereof combined with chemotherapy and/or radiotherapy provides additive effects and may be used in the treatment as well as in the prophylaxis of recurrence of cancer. It has surprisingly been discovered that a method of the invention decreases the probability of long-term recurrence of cancer and provides loco-regional control.

There appears to be an additive or synergistic effect by combining administration of nimorazole or a pharmaceutically acceptable salt thereof and cisplatin with radiotherapy in the treatment and/or prophylaxis of recurrence of SCC.

The claims provide aspects and embodiments of the present invention.

The invention relates to a method for the treatment of cancer, comprising a step of administration of nimorazole, radiotherapy and/or chemotherapy.

According to an aspect, the invention concerns a method for the treatment of cancer and/or prevention of recurrence of cancer and/or prophylaxis of cancer, comprising a step of administration of nimorazole or a pharmaceutically acceptable salt thereof, radiotherapy and/or chemotherapy.

According to another aspect, the invention concerns nimorazole or a pharmaceutically acceptable salt thereof for use in a method of treatment, prevention of recurrence of cancer, and/or prophylaxis according to the invention.

According to another aspect, the invention concerns a pharmaceutical composition comprising nimorazole or a pharmaceutically acceptable salt thereof for use in a method of treatment, prevention of recurrence of cancer, and/or prophylaxis according to the invention.

### BRIEF DESCRIPTION OF THE DRAWINGS

FIG. 1 shows loco-regional failure after treatment of HNSCC with chemo-radiotherapy in combination with nimorazole or placebo, respectively. For more details see example 4.

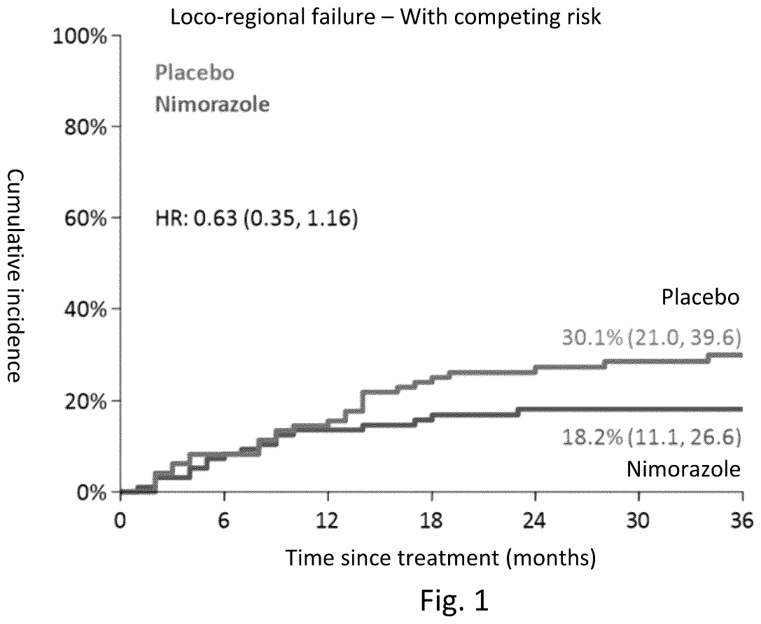

### DETAILED DESCRIPTION

Embodiments of the invention are provided in the claims.

According to an embodiment the invention relates to a method for treating cancer, comprising administering nimorazole in combination with chemotherapy and/or radiotherapy.

Nimorazole (INN) is reported to be a member of imidazoles and a C-nitro compound, with the molecular formula C₉H₁₄N₄O₃. The systematic name is reported to be 4-[2-(5-nitroimidazol-1-yl)ethyl]morpholine.

According to an embodiment of the invention Nimorazole is administered to a patient suffering from cancer in combination with chemotherapy and/or radiotherapy.

In general, nimorazole is administered in a pharmaceutical composition comprising nimorazole and one or more pharmaceutically acceptable excipients as known in the art.

The invention is not limited to any particular administration form, but any known pharmaceutical composition comprising nimorazole may be used, even though it is preferred to administer nimorazole as a tablet, preferably a coated tablet and most preferred using a dispersible tablet as disclosed in WO 2014/075692.

Patients to be treated according to the invention are human beings diagnosed with cancer, in particular where the cancerous tumor exhibits hypoxia.

Hypoxia is characterized as a condition in which the oxygen availability to the tissue in question is low. Hypoxia in tumors may be a consequence of high metabolic activity in the tumor cells leading to an oxygen demand that exceeds the amount that is delivered to the tissue by blood circulation. As a consequence of the hypoxia, cells in the inner of a hypoxic tumor will have a lower metabolic activity than would have been possible if the supply of oxygen was adequate.

Other hypoxic cancers are cancers that have developed in tissues that are located a long distance from the blood vessels providing for the oxygen supply, such as cancers in the mucosa or the skin. This is because the mucosa and the skin consist of many layers of cells separating the inner tissues from the surroundings. Parts of these tissues are separated from the blood vessels by several layers of cells and are therefore primed to thrive under hypoxic conditions and will therefore keep such capabilities when eventually transforming to malignant cells.

It has been observed by several researchers within the field that hypoxic tumors are less susceptible to treatment with radiation or chemotherapeutic compounds.

In general it may be assumed that cancerous tumors are hypoxic.

The hypoxic status of a tumor can be determined by taking a biopsy from the tumor and determine the hypoxia status by analysing the active expression of one or more genes that are predictive for hypoxia. A 15 gene expression classifier test for detecting hypoxia has been developed (Toustrup K, Sorensen B S, Nordsmark M, Busk M, Wiuf C, Alsner J et al. Development of a hypoxia gene expression classifier with predictive impact for hypoxic modification of radiotherapy in head and neck cancer. Cancer Res. 2011:71(17)5923-31.) and the classifier test has been clinically validated (Toustrup K, Sorensen B S, Metwally M A. Validation of a 15-gene hypoxia classifier in head and neck cancer for prospective use in clinical trials. Acta Oncol. 2016:(55):1091-8).

In one preferred embodiment the patient to be treated according to the present invention is a patient having a hypoxic tumor, where the hypoxia of the tumor has been confirmed using a gene expression classifier test, even more preferred, wherein the hypoxia of the tumor has been confirmed using the 15-gene hypoxia classifier as referred to above.

According to an embodiment, the cancer to be treated according to the method of the invention may be any cancer forming or suspected of forming hypoxic cancers.

Examples of cancer that may be treated according to the invention include cancer selected among breast cancer, head/neck cancer, esophagus cancer, lymphoma, cervical cancer, anal cancer, brain cancer, lung cancer, bladder cancer, and prostate cancer.

According to an embodiment, a preferred cancer for treatment according to the invention is a HNSCC, in particular selected among laryngeal, hypopharyngeal and/or oropharyngeal squamous cell carcinoma (SCC).

According to an embodiment of the invention nimorazole is administered in an amount in the range of 600-3000 mg pr m² body surface, preferably in the range of 900-1500 mg pr m² body surface and most preferred approximately 1200 mg per m² body surface. In case of an accelerated radiotherapy regimen, with more than one radiation treatment per day, a dosage of only 1000 mg nimorazole independent of body surface may be administered before the second and subsequent radiation treatment same day.

According to an embodiment of the invention nimorazole is administered up to 180 minutes before the radiotherapy, preferably between 60 and 120 minutes before the radiotherapy and most preferred approximately 90 minutes before radiotherapy.

The radiation used according to an embodiment of the invention may be delivered using any device for radiotherapy as known in the field.

In one embodiment the radiotherapy is delivered using intensity-modulated radiation therapy (IMRT).

The treatment according to an embodiment of the invention is typically performed over a period extending over several weeks and comprising several administrations of nimorazole shortly before the discrete radiotherapy treatments, where the chemotherapeutic agent is administered concomitantly during the treatment period. The whole treatment period will be selected by the doctor responsible and is typically in the range of 4 weeks to 12 weeks, such as 4, 5, 6, 7, 8, 9, 10, 11 or 12 weeks. Some treatment regimen may even comprise two or more separate treatment periods.

The radiotherapy may be delivered using an intensity of up to 90 Gy over a treatment period of up to 10 weeks, such as 9 weeks, 8 weeks, 7 weeks, 6 weeks, 5 weeks, 4 weeks or even shorter, preferably about 70 Gy in 6 weeks.

The radiotherapy is conveniently delivered in a series of discrete radiation treatments (fractions) distributed over the complete treatment period, such as a daily radiotherapy treatment delivering 1-4 Gy, preferably 2 Gy. In some embodiments, in particular for accelerated treatment regimen the patient may receive more than one discrete fraction pr day, typically 2 or 3 fractions.

According to an embodiment the invention is not limited to any particular chemotherapeuticals, but the combined treatment using nimorazole administration, radiotherapy and a chemotherapeutic agent may in principle use any chemotherapeutic agent as known in the art.

A preferred chemotherapeutic agent is cisplatin. Cisplatin may according to the invention be administered in a total amount of up to 100 mg/m², 150 mg/m², 200 mg/m², 250 mg/m² or 300 mg/m². A total amount of 200 mg/m² is preferred.

Cisplatin may be administered in several administration regimens e.g., administered weekly, biweekly or 3-weekly.

In a preferred treatment regimen with a treatment period of 5 weeks or longer, the administration of cisplatin may be delivered as a weekly amount of 40 mg/m² in week 1 to 5 or as an administration of 100 mg/m² in weeks 1 and 4.

In one preferred embodiment the method of the invention is used for the treatment of HNSCC in an accelerated radiotherapy procedure, having a total treatment period of 6 weeks, and comprises
a total radiation of 70 Gy, delivered as 35 fractions, with delivery of 6 fractions of 2 Gy pr week until the total radiation has been delivered;
1.2 g/m² nimorazole administered 90 min (+/−30 min) before each radiotherapy fraction; and
cisplatin delivered as a weekly amount of 40 mg/m² in week 1 to 5 or as an administration of 100 mg/m² in weeks 1 and 4.

According to an embodiment a method of the invention provides for an improved treatment of cancer compared with a similar treatment method without administration of nimorazole. Without wishing to be bound by any theory, it is believed that nimorazole administration results in the hypoxic tumors becoming more susceptible to cancer treatment by radiotherapy and/or chemotherapy.

Further, it may been shown that a method of the invention may result in prevention of recurrence of the cancer in long periods e.g. for at least 1 month, preferably 3 months, more preferred 6 months, preferably 9 months, more preferred 12 months, preferably 18 months, more preferred 24 months, more preferred 30 months, preferably 36 months, more preferred 42 months, preferably 48 months, more preferred 60 months, preferably 66 months, more preferred 72 months, preferably 10 years.

According to an embodiment, the invention concerns a method for the treatment of cancer and/or prevention of recurrence of cancer and/or prophylaxis of cancer, comprising a step of administration of nimorazole or a pharmaceutically acceptable salt thereof, radiotherapy and optionally chemotherapy.

According to an embodiment, the invention concerns the method comprising chemotherapy.

According to an embodiment, the invention concerns the method comprising a step of administration of nimorazole or a pharmaceutically acceptable salt thereof and chemotherapy. According to this embodiment, chemotherapy and nimorazole may have a synergistic effect even in the absence of radiotherapy. Without being bound by theory, it may be speculated that chemotherapy, such as the administration of cisplatin, may be less efficacious with respect to hypoxic cells, and nimorazole may increase the efficacy with respect to hypoxic cells.

According to an embodiment, the invention concerns the method for the treatment of cancer.

According to an embodiment, the invention concerns the method for prevention of recurrence of cancer.

According to an embodiment, the invention concerns the method according to any of the preceding claims for the prophylaxis of cancer, such as for patients undergoing treatment for cancer.

According to an embodiment, the invention concerns the method, wherein the recurrence of cancer is prevented for at least 1 month, preferably 3 months, more preferred 6 months, preferably 9 months, more preferred 12 months, preferably 18 months, more preferred 24 months, more preferred 30 months, preferably 36 months, more preferred 42 months, preferably 48 months, more preferred 60 months, preferably 66 months, more preferred 72 months, preferably 10 years.

According to an embodiment, the invention concerns the method, wherein said step of chemotherapy comprises administration of an active ingredient selected among cisplatin, carboplatin, ormaplatin, oxaliplatin, DWA2114R, enloplatin, lobaplatin, CI-973 (NK-121), 254-S, JM-216, liposome-entrapped cis-bis-neodecanoato-trans-R,R-1,2-diaminocyclohexane platinum (II) (LNDDP) docetaxel, paclitaxel, capecitabine, flourouracil, and gemcitabine; and a mixture of any of these.

According to an embodiment, the invention concerns the method, wherein said step of chemotherapy comprises administration of an active ingredient selected among cisplatin, carboplatin, ormaplatin, oxaliplatin, DWA2114R, enloplatin, lobaplatin, CI-973 (NK-121), 254-S, JM-216, and liposome-entrapped cis-bis-neodecanoato-trans-R,R-1,2-diaminocyclohexane platinum (II) (LNDDP); and a mixture of any of these.

According to an embodiment, the invention concerns the method, wherein said step of chemotherapy comprises administration of an active ingredient selected among cisplatin, carboplatin, and oxaliplatin; and a mixture of any of these.

According to an embodiment, the invention concerns the method, wherein said step of chemotherapy comprises administration of an active ingredient selected among docetaxel, paclitaxel, capecitabine, fluorouracil, and gemcitabine; and a mixture of any of these.

According to an embodiment, the invention concerns the method, wherein said step of chemotherapy comprises administration of cisplatin.

According to an embodiment, the invention concerns the method, wherein the total amount of cisplatin administered is selected among 50-1000, 100-500, 150-250, and 200 mg/m², preferably 200 mg/m².

According to an embodiment, the invention concerns the method, wherein cisplatin is administered weekly, bi-weekly, 3-weekly, 4-weekly, 5-weekly or 6-weekly, preferably weekly or 3-weekly.

According to an embodiment, the invention concerns the method, wherein cisplatin is administered in a weekly amount of 40 mg/m² on week 1 to 5; or 3-weekly in an amount of 100 mg/m² on weeks 1 and 4.

According to an embodiment, the invention concerns the method, wherein nimorazole or a pharmaceutically acceptable salt thereof is administered orally.

According to an embodiment, the invention concerns the method, wherein a daily dose of nimorazole or a pharmaceutically acceptable salt thereof of 0.1-10, 0.3-5, 0.5-2.5, 0.7-2, 1-1.5, or 1.2 mg/m2, preferably 1.2 mg/m2, is administered.

According to an embodiment, the invention concerns the method, comprising administering nimorazole or a pharmaceutically acceptable salt thereof 10-240, 20-120, or 30-60 minutes before radiotherapy, such as irradiation treatment.

According to an embodiment, the invention concerns the method, wherein the radiotherapy comprises irradiation therapy.

According to an embodiment, the invention concerns the method, wherein the radiotherapy is for curative treatment or reirradiation of cancer.

According to an embodiment, the invention concerns the method, wherein the radiotherapy comprises 1st line irradiation treatment of cancer with curative intent or 2nd line reirradiation treatment of cancer with curative and/or palliation intent.

According to an embodiment, the invention concerns the method, wherein the radiotherapy is delivered using intensity-modulated radiation therapy (IMRT).

According to an embodiment, the invention concerns the method, wherein the radiotherapy is delivered during a period selected among 1-52, 2-26, 3-14, 4-10, 5-8, and 6 weeks. According to an embodiment, the invention concerns the method, wherein the radiotherapy is delivered using an intensity selected among 5-500, 10-300, 20-200, 30-150, 40-125, 50-100, 60-80, and 70 Gy.

According to an embodiment, the invention concerns the method, wherein the radiotherapy is delivered using an intensity of 70 Gy in 6 weeks.

According to an embodiment, the invention concerns the method, wherein the cancer is to some degree a hypoxic cancer or wherein the cancer may be hypoxic.

According to an embodiment, the invention concerns the method, wherein the cancer is a hypoxic cancer.

According to an embodiment, the invention concerns the method, wherein the cancer is a squamous cell carcinoma (SCC).

According to an embodiment, the invention concerns the method, wherein the cancer is selected among breast cancer, head/neck cancer, esophagus cancer, lymphoma, anal cancer, cervix or cervical cancer, brain cancer, lung cancer, bladder cancer, and prostate cancer.

According to an embodiment, the invention concerns the method, wherein the cancer is head/neck cancer.

According to an embodiment, the invention concerns the method, wherein the cancer is cervical cancer or lung cancer, such as inoperable lung cancer.

According to an embodiment, the invention concerns the method, wherein the administration of nimorazole or a pharmaceutically acceptable salt thereof comprises administration of a tablet comprising nimorazole or a pharmaceutically acceptable salt thereof or administration of an aqueous solution obtained by dissolving a tablet comprising nimorazole or a pharmaceutically acceptable salt thereof in water.

According to an embodiment, the invention concerns the method, wherein the administration of nimorazole or a pharmaceutically acceptable salt thereof comprises use of a coated tablet.

According to an embodiment, the invention concerns the method, wherein the administration of nimorazole or a pharmaceutically acceptable salt thereof comprises use of a tablet comprising at least 250 mg nimorazole or a pharmaceutically acceptable salt thereof.

According to an embodiment, the invention concerns the method, wherein the administration of nimorazole or a pharmaceutically acceptable salt thereof comprises use of a tablet comprising 100-2000 mg, preferably 300-1500 mg, more preferred 400-1000 mg, preferably 500 mg nimorazole or a pharmaceutically acceptable salt thereof.

According to an embodiment, the invention concerns the method, wherein the administration of nimorazole or a pharmaceutically acceptable salt thereof comprises oral administration or administration by a feeding tube.

According to an embodiment, the invention concerns nimorazole or a pharmaceutically acceptable salt thereof for use in a method of treatment, prevention of recurrence of cancer, and/or prophylaxis according to the invention.

According to an embodiment, the invention concerns a pharmaceutical composition comprising nimorazole or a pharmaceutically acceptable salt thereof for use in a method of treatment, prevention of recurrence of cancer, and/or prophylaxis according to the invention.

According to an embodiment, the invention concerns the nimorazole or a pharmaceutically acceptable salt thereof or pharmaceutical composition according to the invention, wherein the chemotherapy comprises administration of cisplatin.

All cited references are incorporated by reference.

The accompanying FIGURES, Examples, and Claims are provided to explain rather than limit the present invention. It will be clear to the person skilled in the art that aspects, embodiments, claims and any items of the present invention may be combined.

Unless otherwise mentioned, all percentages are in weight/weight. Unless otherwise mentioned, all measurements are conducted under standard conditions (ambient temperature and pressure). Unless otherwise mentioned, test conditions are according to European Pharmacopoeia 8.0.

### EXAMPLES

### Example 1—DAHANCA 5 Clinical Study

A double-blinded randomized prospective phase III study treating HNSCC with Nimorazole and radiotherapy was conducted and completed in Denmark. The study included 414 eligible patients and showed a significantly improved loco-regional control rate (49 versus 33%, p=0.002) (HR 0.69, Cl: 0.53, 0.90) when conventional primary radiotherapy was supplemented with concurrent nimorazole compared with placebo (no nimorazole). Nimorazole was administered 90 minutes (+/−30 minutes) prior to the first of each daily radiotherapy treatment in a dose of approximately 1.2 g/m² body surface. In case of accelerated radiotherapy regimen, the second daily dose of only 1 g (1,000 mg) was administered independently of body surface. The total dose during the radiotherapy course was intended to amount to approximately 36 g (3,600 mg)/m² and not exceed 40 g/m² or a total of 75 g. This dose was expected to result in maximum radiotherapy enhancement ratio and represented the maximal tolerable dose. Disease specific survival was also significantly improved (HR 0.76, CI: 0.58, 0.99), whereas only an insignificant trend towards improved overall survival was found.

### Example 2—IAEA-HypoX Study

The IAEA-HypoX study included patients with HNSCC treated with accelerated radiotherapy and randomized to +/− concurrent nimorazole. Due to logistic problems the study was terminated prematurely, but analysis of the 84 enrolled patients showed a non-significant tendency towards beneficial locoregional control in the patients treated with concurrent nimorazole (HR: 0.72, CI: 0.38-1.37).

### Example 3—NIMRAD Study

NIMRAD included 338 HNSCC patients not fit for concurrent chemotherapy to be randomized to radiotherapy+/− nimorazole. Also, in this study a non-significant tendency towards beneficial locoregional control in the patients treated with concurrent nimorazole was observed (HR: 0.76, CI: 0.48-1.21).

### Example 4—EORTC 1219 Study

EORTC 1219 investigated nimorazole given to patients treated with accelerated fractionation and concurrent cisplatin.

Patients were randomized in two groups receiving either nimorazole (NIM) or placebo, given concomitantly with chemo-radiotherapy. Accelerated radiotherapy was delivered using IMRT (70 Gy in 6 weeks). Total Cisplatin dose was 200 mg/m² either weekly (40 mg/m² on week 1 to 5) or 3-weekly (100 mg/m² on weeks 1 & 4). NIM or placebo were delivered orally with a daily dose of 1.2 mg/m². The primary endpoint was 3-year locoregional failure.

Secondary endpoints included disease specific survival (DSS), overall survival (OS), and acute and late morbidity. A final institutional revision of the tumor and survival data was recently performed. Also, this study was prematurely closed, primarily due to low recruitment and economic issues. A total of 194 patients were included and the study showed a 3 years locoregional control in favor of the patients having nimorazole (HR: 0.63, CI: 0.35-1.15). Despite the reduced patient number, NIM was found to improve locoregional control in advanced HNSCC, when added to the chemoradiation regimen given to the patients, but without significant improvement in DSS or OS.

### Example 5—Combined Studies

Conclusively a range of studies with radiotherapy treated HNSCC+/− nimorazole all show a clear and comparable tendency in favour of supplemental hypoxic modification with nimorazole (HR^{~}0.7). As the majority of these above-mentioned studies did not attain enough power to individually show significant difference, a meta-analysis focusing distinctly on the relevance of hypoxic modification with nimorazole in the radiotherapeutic treatment of HNSCC has been performed based on the 4 randomized trials. The meta-analysis showed beneficial effect of adding nimorazole in terms of locoreginal control (HR: 0.70 (0.57-0.86), see FIGURE, disease specific survival (HR: 0.79 (0.64-0.97) but not overall survival (HR: 0.95 (0.79-1.12). The meta-analysis did also demonstrate that the four trials were very homogeneous in their outcome.

TABLE 1
Meta-analysis Nimorazole in radiotherapeutic treatment of HNSCC

| Study | Planned | Included | HR (95% CI) |
|---|---|---|---|
| DAHANCA 5 | 400 | 414 | 0.69 (0.53, 0.90) |
| IAEA-HypoX | 600 | 84 | 0.72 (0.38, 1.37) |
| EORTC 1219/DAHANCA 29 | 640 | 194 | 0.63 (0.35, 1.15) |
| NIMRAD | 470 | 338 | 0.76 (0.48, 1.21) |
| Overall | 2.110 | 1.030 | 0.70 (0.57, 0.86) |

Cochran's Q: p = 0.97;
I² < 0.01%

1. A method for the treatment of cancer or decreasing the probability of recurrence of cancer for at least 36 months, comprising administering to a patient in need thereof (i) nimorazole or a pharmaceutically acceptable salt thereof, (ii) radiotherapy, and (iii) chemotherapy comprising administration of cisplatin.

2. The method according to claim 1 which is for the treatment of cancer.

3. The method according to claim 1 which is for decreasing the probability of recurrence of cancer.

4. The method according to claim 3, wherein the patient is undergoing treatment for cancer.

5. The method according to claim 3, wherein the probability of recurrence of cancer is decreased for at least 48 months.

6. The method according to claim 1, wherein said chemotherapy further comprises administration of an active ingredient selected from the group consisting of carboplatin, ormaplatin, oxaliplatin, DWA2114R, enloplatin, lobaplatin, CI-973 (NK-121), 254-S, JM-216, liposome-entrapped cis-bis-neodecanoato-trans-R,R-1,2-diaminocyclohexane platinum (II) (LNDDP), docetaxel, paclitaxel, capecitabine, flourouracil, gemcitabine, and a mixture of any of these.

7. The method according to claim, wherein the total amount of cisplatin administered is 50-1000 mg/m².

8. The method according to claim 1, wherein cisplatin is administered weekly.

9. The method according to claim 8, wherein cisplatin is administered in a weekly amount of 40 mg/m² on week 1 to 5.

10. The method according to claim 1, wherein the nimorazole or pharmaceutically acceptable salt thereof is administered orally.

11. The method according to claim 1, wherein the nimorazole or pharmaceutically acceptable salt thereof is administered as a daily dose of 0.1-10 mg/m².

12. The method according to claim 1, wherein the nimorazole or pharmaceutically acceptable salt thereof is administered 10-240 minutes before administering the radiotherapy.

13. The method according to claim, wherein the radiotherapy is for curative treatment of cancer or reirradiation of cancer.

14. The method according to claim, wherein the radiotherapy comprises 1st line irradiation treatment of cancer with curative intent or 2nd line reirradiation treatment of cancer with curative and/or palliation intent.

15. The method according to claim 1, wherein the radiotherapy is delivered using intensity-modulated radiation therapy (IMRT).

16. The method according to claim 1, wherein the radiotherapy is delivered during a period of 1-52 weeks.

17. The method according to claim 1, wherein the radiotherapy is delivered using an intensity of 5-500 Gy.

18. The method according to claim 1, wherein the radiotherapy is delivered using an intensity of 70 Gy over 6 weeks.

19. The method according to claim 1, wherein the cancer is, or is suspected of being, at least in part a hypoxic cancer.

20. The method according to claim 1, wherein the cancer is a hypoxic cancer.

21. The method according to claim 1, wherein the cancer is a squamous cell carcinoma (SCC).

22. The method according to claim 1, wherein the cancer is selected from the group consisting of breast cancer, head/neck cancer, esophageal cancer, lymphoma, anal cancer, cervix or cervical cancer, brain cancer, lung cancer, bladder cancer, and prostate cancer.

23. The method according to claim 1, wherein the cancer is head/neck cancer.

24. The method according to claim 1, wherein the cancer is cervical cancer or lung cancer.

25. The method according to claim 1, wherein the administration of nimorazole or a pharmaceutically acceptable salt thereof comprises administration of a tablet comprising nimorazole or a pharmaceutically acceptable salt thereof or administration of an aqueous solution obtained by dissolving a tablet comprising nimorazole or a pharmaceutically acceptable salt thereof in water.

26. The method according to claim 1, wherein the administration of nimorazole or a pharmaceutically acceptable salt thereof comprises use of a coated tablet.

27. The method according to claim 1, wherein the administration of nimorazole or a pharmaceutically acceptable salt thereof comprises use of a tablet comprising 100-2000 mg nimorazole or a pharmaceutically acceptable salt thereof.

28. The method according to claim 1, wherein the method is for the treatment of head and neck squamous cell carcinomas (HNSCC); wherein the total radiation dose is 70 Gy, delivered as 35 fractions, with delivery of 6 fractions of 2 Gy pr week until the total radiation has been delivered; wherein 1.2 g/m² nimorazole is administered at 90 min (+/−30 min) before each radiotherapy fraction; and wherein cisplatin is administered as a weekly amount of 40 mg/m² in weeks 1 to 5, or as an administration of 100 mg/m² in weeks 1 and 4.

29. The method according to claim 1, wherein the administration of nimorazole or a pharmaceutically acceptable salt thereof comprises oral administration or administration by a feeding tube.
